(19) United States
(12) Patent Application Publication
(10) Pub. No.: US 2009/0155236 A1
(43) Pub. Date: Jun. 18, 2009

(54) Peptide sequence for modulation of delta protein kinase c

(75) Inventors: Daria D. Mochly-Rosen, Menlo Park, CA (US); Viktoria Kheifets, Mountain View, CA (US); Deborah Schechtman, Ipanema (BR); Koichi Inagaki, Shiga (JP); Yasuki Kihara, Otsu City (JP)

Correspondence Address: PERKINS COIE LLP, P.O. BOX 1208, SEATTLE, WA, 98111-1208, US

(73) Assignee: NATIONAL INSTITUES OF HEALTH (NIH), U.S. DEPT OF HEALTH AND HUMAN SERVICE U.S. GOVERNMENT, BEHTESDA (MD)

(21) Appl. No.: 11/659,305

(22) PCT Filed: Aug. 2, 2005
(86) PCT No.: PCT/US05/27567

§ 371 Date: Dec. 15, 2008

Related U.S. Application Data
(60) Provisional application No. 60/598,401, filed on Aug. 2, 2004.
(60) Provisional application No. 60/598,458, filed on Aug. 2, 2004.

(51) Int. Cl. A61K 38/45; C12N 9/12

(52) U.S. Cl. 424/94.5; 435/194

(57) ABSTRACT

A peptide identified from the annexin V protein sequence and having the ability to modulate the activity of delta protein kinase C (PKC) is described. The peptide and variants thereof bind to delta PKC and are effective to regulate delta PKC mediated cellular responses. In particular the annexin V peptide is able to inhibit protein-protein interaction with delta PKC, blocking intracellular translocation of delta PKC. Inhibition of delta PKC translocation confers protection to tissue at risk of exposure to ischemia or hypoxia or to reperfusion injury.

### TECHNICAL FIELD

This invention relates to amino acid sequences of annexin V and to the use of these sequences in the regulating cellular responses mediated by delta protein kinase C.

### BACKGROUND

Many biological processes involve specific protein-protein interactions. Protein-protein interactions, which may be transient or otherwise, enable two or more proteins or subunits to associate. Protein-protein interactions can have a number of measurable effects: they can alter kinetic properties of proteins, they are a common mechanism for allowing substrate channeling; they can result in the formation of new binding sites; they can alter the activity of a protein, and/or alter the specificity of a protein for its substrate (Phizicky and Fields, Microbiological Reviews, 59:94-123, (1995); Pawson and Nash, Gene Dev., 14:1027-1047, (2000)).

The protein kinase C (PKC) family consists of several lipid-activated isozymes playing key roles in many signal transduction pathways. Three groups of PKC have been distinguished: the conventional, calcium-, and phospholipid-, and diacylglycerol (DAG)-dependent isoenzymes alpha (α), beta (β), and gamma (γ); the novel forms, delta (δ), epsilon (ε), theta (θ), and eta (η), which are calcium-independent; and the atypical isoenzymes zeta (ζ) and iota (ι) which are both calcium- and DAG-independent. The isoenzymes exhibit different tissue distributions and activator requirements, and play individual roles in intracellular signaling. Activation of PKCs often involves translocation from cytosolic to membrane compartments or between different intracellular locations, and when a number of isozymes are translocated upon cell stimulation, different redistributions have been observed. This suggests isozyme-specific interaction with membrane and cytoskeletal proteins both before and upon PKC activation, and such divergence may enable phosphorylation of different proteins, already compartmentalized at the various sites.

A number of proteins which interact with PKCs have been reported. For example, inactive PKC may be localized by scaffolding proteins such as AKAP 79, and released upon lipid hydrolysis to phosphorylate co-localized proteins (Klauck, T. et al., Science, 271:1589 (1996)). Scaffolding proteins may also co-ordinate the actions of other kinases and phsophatases to promote cross-talk and signal termination. Proteins which bind PKC in a phospholipid-dependent manner have been described, such as receptors for activated C-kinase (RACKs) (Mochly-Rosen, D., Science, 268:247 (1995) and cytoskeletal proteins such as vinculin and talin (Jaken, S. Curr. Opin Cell Biol., 8:168 (1996)). Some RACKs are isozyme specific and use of RACK-derived peptides to block individual isozyme relocalization interferes with specific cell functions (Yedovitzky, M. et al., J. Biol. Chem., 272:1417 (1997); Johnson, J. A. et al., J. Biol. Chem., 271:24962 (1996)).

Annexins are a family of about ten structurally related proteins found in diverse eukaroytic organisms such as fruit fly, sponges, slime molds, higher plants, and mammals (Towle, C. A. et al., J. Biol. Chem., 267:5416 (1992)). Proteins in this family reversibly bind to negatively charged phospholipids (phosphatidylcholine and phosphatidylserine) in a calcium dependent manner. Many of the functions attributed to annexins are believed to be the result of this binding property. These functions include; (1) regulation of phospholipase A2 activity, (2) anticoagulant activity, (3) roles in cellular exocytosis, (4) membrane trafficking, (5) cytoskeletal organization, (6) phosphohydrolase activity, (7) various aspects of cell proliferation, and (8) calcium channel activity (Towle et al., Id.).

Annexin V is a specific family member found in a variety of species including human. It is widely distributed in various cells and tissues and is particularly abundant in brain, where it is believed to act as a paracrine-type neurotrophic factor (Ohsawa, K. et al. J. Neurochem., 67:89 (1996)). It is also known to possess anticoagulant activity, transport Ca²⁺ ions across phospholipid membranes, and inhibit phospholipase A2.

A link between PKC-alpha and annexin VI from skeletal muscle has been reported (Schmitz-Peiffer, C., et al., Biochem. J., 330:675 (1998)). Also, annexins I, II, and IV have been found to be substrates for PKC in vitro (Varticovski, L. et al., Biochemistry, 27:3682 (1988); Summers, T. A. et al., J. Biol. Chem., 260:2437 (1985); Weber, K. et al., EMBO J., 6:1599 (1987)).

To date, there has been no link between annexin V and delta PKC. It is known that delta PKC is involved in tissue damage during ischemia and/or reperfusion. More specifically, inhibition of delta PKC by administering a delta PKC peptide inhibitor (antagonist) during simulated ischemia/reperfusion in isolated rat hearts is cardioprotective (Inagaki, K. et al., Circulation, 108(19):2304 (2003); Inagaki, K. et al., Circulation, 108(7):869 (2003)). There remains a need in the art for therapeutic agents capable of regulating the activity of delta PKC, particularly for modulating the activity of delta PKC in its role associated with ischemia, in order to reduce the deleterious effects of ischemia and of reperfusion injury.

The foregoing examples of the related art and limitations related therewith are intended to be illustrative and not exclusive. Other limitations of the related art will become apparent to those of skill in the art upon a reading of the specification and a study of the drawings.

### SUMMARY

In a first aspect, a substantially pure, isolated or recombinant polypeptide is provided, the polypeptide selected from the group consisting of: (i) the amino acid sequence comprising or consisting of the sequences identified herein as SEQ ID NO:1 or SEQ ID NO:11; and (ii) a variant having one or more amino acid substitutions, modifications, deletions, or insertions relative to the amino acid sequence identified as SEQ ID NO:1, the variant being at least about 50% identical to SEQ ID NO:1 and retaining at least a proportion of the activity of SEQ ID NO:1.

In another aspect, pharmaceutical compositions comprising one or more of these peptides are provided. The compositions are suitable for use in treating or preventing tissue damage due to an ischemic or hypoxic event or due to reperfusion injury, for providing preconditioning protection, for regulating cellular processes mediated by delta PKC.

In another aspect, methods for treating or preventing tissue damage due to an ischemic or hypoxic event or due to reperfusion injury are described. In the methods, an effective amount of a pharmaceutical composition comprising a substantially purified polypeptide having the amino acid sequence shown in SEQ ID NO:1 or a variant thereof is administered to a subject in need of such treatment.

In another aspect, methods for preconditioning tissue that is at risk or ischemia or reperfusion injury, by administering one of the aforementioned peptides, are described.

In still another aspect, methods for modulating the interaction of annexin V and delta PKC by administering one of the aforementioned peptides are provided.

These and other objects and features of the invention will be more fully appreciated when the following detailed description of the invention is read in conjunction with the accompanying drawings.

### BRIEF DESCRIPTION OF THE DRAWINGS

FIG. 1 shows the results from an overlay assay to evaluate binding of delta PKC, the delta V1 domain of PKC, the delta V5 domain of delta PKC, and epsilon PKC to annexin V protein, where Western blots were probed for the respective PKC isozymes;

FIG. 2 shows the percent binding of the indicated domains, epsilon V1 PKC and delta V1 PKC to annexin V protein, in the presence and absence of calcium;

FIG. 3 shows the percent binding of annexin 1 and annexin V to delta PKC in the presence and absence of calcium;

FIG. 4A is an SDS-PAGE gel of an in vitro kinase reaction with histone 2A as substrate conducted with delta PKC alone (Lane 1), in the presence of phosphatidylserine (PS) and diacylglycerol (DAG; Lane 2), in the presence of PS/DAG and annexin V (Lane 3); in the presence of PS/DAG and JTV-519 (Lane 4); in the presence of PS/DAG, JTV-519 and annexin V (Lane 5);

FIG. 4B is an SDS-PAGE gel of an in vitro kinase reaction with histone 2A as substrate conducted with epsilon PKC alone (Lane 2), in the presence of phosphatidylserine (PS) and diacylglycerol (DAG; Lane 1), and in the presence of PS/DAG and annexin V (Lane 3);

FIG. 5 is an SDS-PAGE gel of immuno-precipitated cross-linked delta PKC probed with anti-annexin V, the cells prior to lysing stimulated with phorbol myristate acetate (PMA), hydrogen peroxide (H₂O₂), JTV-519, or delta V1-1 peptide and JTV-519, for times of between 30 seconds to 20 minutes;

FIGS. 6A-6B show an SDS-PAGE gel showing the co-immunoprecipitation of annexin V and delta PKC in cells (FIG. 6A) and a corresponding bar graph of maximum percent association of annexin V and delta PKC (FIG. 6B), after stimulation of cells with PMA or treatment with JTV-519;

FIG. 6C is an SDS-PAGE gel of fractionated PMA-stimulated cell lysates probed with anti-delta PKC;

FIG. 6D, is an SDS-PAGE gel of cross-linked, fractionated cell lysates pulled down with anti-delta PKC and probed with anti-annexin V;

FIG. 7 shows a series of SDS-PAGE gels illustrating dissociation of delta-PKC-annexin V complex in the presence of deoxyglucose, nocodazole, and cytochalasin;

FIGS. 8A-8B show an SDS-PAGE gel showing the translocation of delta PKC from the soluble to the membrane fraction of CHO cells (FIG. 8A) and a corresponding bar graph fraction of delta PKC protein in the membrane fraction relative to the total protein, for cells untreated and for cells exposed to TAT-AnxV peptide and to TAT-AnxV_{(R→E)} (denoted in the figures as “pAnx” and “EpAnxV”, respectively) and stimulated with PMA;

FIGS. 9A-9B are bar graphs showing the infarct size, in percent, (FIG. 9A) and the amount of creatine phosphokinase (CPK) released, in U, (FIG. 9B) in isolated rat hearts ex vivo subjected to ischemia/reperfusion, and left untreated or treated prior to ischemia with TAT-AnxV peptide or TAT-AnxV_{(R→E)} (denoted in the figures as “pAnx” and “EpAnxV”, respectively); and

FIGS. 10A-10B are bar graphs showing the infarct size, in percent, (FIG. 10A) and the amount of creatine phosphokinase (CPK) released, in U, (FIG. 10B) in isolated rat hearts ex vivo subjected to ischemia/reperfusion, and left untreated or treated after ischemia and during the initial period of reperfusion, with TAT-AnxV peptide or TAT-AnxV_{(R→E)} (denoted in the figures as “pAnx” and “EpAnxV”, respectively).

### BRIEF DESCRIPTION OF THE SEQUENCES

SEQ ID NO:1 is a peptide from annexin V, residues 157-164, also referred to herein as ‘the annexin V peptide.’

SEQ ID NO:2 is the amino acid sequence of human annexin V protein (“annexin V protein”).

SEQ ID NO:3 is the amino acid sequence of residues 1-141 from the V1 domain of rat delta PKC (accession no. KIRTCD).

SEQ ID NO:4 is the amino acid sequence for the V1 region of delta PKC.

SEQ ID NO:5 is the amino acid sequence for the V5 region of delta PKC.

SEQ ID NO:6 is the amino acid sequence of human epsilon PKC.

SEQ ID NO:7 is the amino acid sequence of the V1 region of human epsilon PKC:

SEQ ID NO:8 is the amino acid sequence of the V5 region of epsilon PKC.

SEQ ID NO:9 is the amino acid sequence of human annexin I protein.

SEQ ID NO:10 is an amino acid sequence from delta PKC (amino acid residues 74-81) and is referred to herein as “pseudo-delta” RACK, or T8 RACK.

SEQ ID NO:11 is a peptide variant of SEQ ID NO:1.

SEQ ID NO:12 is a peptide variant of SEQ ID NO:1.

SEQ ID NO:13 is a peptide variant of SEQ ID NO:1.

SEQ ID NO:14 is a peptide variant of SEQ ID NO:1.

SEQ ID NO:15 is a peptide variant of SEQ ID NO:1.

SEQ ID NO:16 is a peptide variant of SEQ ID NO:1.

SEQ ID NO:17 is a peptide variant of SEQ ID NO:1.

SEQ ID NO:18 is a peptide variant of SEQ ID NO:1.

SEQ ID NO:19 is a peptide variant of SEQ ID NO:1.

SEQ ID NO:20 is a peptide variant of SEQ ID NO:1.

SEQ ID NO:21 is a peptide variant of SEQ ID NO:1.

SEQ ID NO:22 is a peptide variant of SEQ ID NO:1.

SEQ ID NO:23 is a peptide variant of SEQ ID NO: 1.

SEQ ID NO:24 is a peptide variant of SEQ ID NO:1.

SEQ ID NO:25 is a peptide variant of SEQ ID NO: 1.

SEQ ID NO:26 is the Drosophila Antennapedia homeodomain-derived carrier peptide.

SEQ ID NO:27 is a Tat-derived carrier peptide (Tat 47-57).

SEQ ID NO:28 is an amino acid sequence from the first variable region of delta PKC (amino acid residues 8-17), and is referred to herein as “delta V1-1” or 6V1-1.

SEQ ID NO:29 is a conjugate of SEQ ID NO: 1 joined to a TAT carrier peptide (SEQ ID NO:27) through an N-terminal cysteine-cysteine bond, also referred to herein as TAT-AnxV.

SEQ ID NO:30 is a modification of SEQ ID NO:1.

SEQ ID NO:31 is a conjugate of SEQ ID NO:30 joined to a TAT carrier peptide (SEQ ID NO:27) through an N-terminal cysteine-cysteine bond, also referred to herein as TAT-AnxV_{(R→E)} (and denoted to in some figures as “EpAnxV”.

### DETAILED-DESCRIPTION OF THE INVENTION

### I. Definitions

It must be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the methodologies which are reported in the publications which might be used in connection with the invention.

Protein sequences are presented herein using the one letter or three letter amino acid symbols as commonly used in the art and in accordance with the recommendations of the IUPAC-IUB Biochemical Nomenclature Commission.

The term “substantially purified”, as used herein, refers to nucleic or amino acid sequences that are removed from their natural environment, isolated or separated, and are at least 60% free, preferably 75% free, more preferably 90% free, and most preferably 95% free from other components with which they are naturally associated.

“Peptide” and “polypeptide” are used interchangeably herein and refer to a compound made up of a chain of amino acid residues linked by peptide bonds. Unless otherwise indicated, the sequence for peptides is given in the order from the amino terminus to the carboxyl terminus.

A “substitution”, as used herein, refers to the replacement of one or more amino acids by different amino acids, respectively.

An “insertion” or “addition”, as used herein, refers to a change in an amino acid sequence resulting in the addition of one or more amino acid residues, as compared to the naturally occurring molecule.

A “deletion”, as used herein, refers to a change in the amino acid sequence and results in the absence of one or more amino acid residues.

A “variant” of a first amino acid sequence refers to a second amino acid sequence that has one or more amino acid substitutions or deletions, relative to the first amino acid sequence.

A “modification” of an amino acid sequence or a “modified” amino acid sequence refers to an amino acid sequence that results from the addition of one or more amino acid residues, to either the N-terminus or the C-terminus of the sequence.

The term “modulate”, as used herein, refers to a change in the activity of delta protein kinase C. For example, modulation may cause an increase or a decrease in protein activity, binding characteristics, or any other biological, functional or immunological properties of delta PKC.

Reference herein to an “amino acid sequence having ‘x’ percent identity” with another sequence intends that the sequences have the specified percent identity, ‘x’, determined as set forth below, and share a common functional activity. To determine the percent identity of two amino acid sequences, the sequences are aligned for optimal comparison purposes (e.g., gaps can be introduced in one or both of a first and a second amino acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 75%, 80%, 85%, 90%, or 95% of the length of the reference sequence. For the relatively short peptide sequences described herein, percent identity is taken as the number of like residues between the first and second sequence relative to the total number of residues in the longer of the first and second sequences. The comparison of sequences and determination of percent identity between two sequences can also be accomplished using a mathematical algorithm. The percent identity between two amino acid sequences can be determined using the Needleman and Wunsch (J. Mol. Biol., 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. The percent identity between two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. Protein sequences can further be used as a “query sequence” to perform a search against public databases; for example, BLAST protein searches can be performed with the XBLAST program, score=50, wordlength=3. See http://www.ncbi.nlm.nih.gov.

“Ischemia” is defined as an insufficient supply of blood to a specific organ or tissue. A consequence of decreased blood supply is an inadequate supply of oxygen and nutrients to the organ or tissue (hypoxia). Prolonged hypoxia may result in injury to the affected organ or tissue.

“Anoxia” refers to a virtually complete absence of oxygen in the organ or tissue, which, if prolonged, may result in death of the organ or tissue.

“Hypoxic condition” is defined as a condition under which a particular organ or tissue receives an inadequate supply of oxygen.

“Anoxic condition” refers to a condition under which the supply of oxygen to a particular organ or tissue is cut off.

“Ischemic injury” refers to cellular and/or molecular damage to an organ or tissue as a result of a period of ischemia.

### II. Peptide and Peptide Compositions

The invention is based on the identification of peptide sequences capable of binding to delta PKC, and thereby capable of regulating delta PKC mediated cellular activities. As noted above, activation of delta-PKC and its translocation to the plasma membrane induces preconditioning of cardiac tissue (Mitchell, M. B. et al. Circ. Res., 76:73 (1995)). Preconditioning of cardiac tissue with brief episodes of ischemia is a potent means of reducing irreversible tissue injury during subsequent prolonged ischemia. As will be illustrated below, the peptide sequences described herein act as pharmacological agents that can induce activation of delta-PKC to exert its anti-ischemic effects. A peptide having the amino acid sequence LQANRDPD (SEQ ID NO:1), also referred to herein as the ‘annexin V peptide’, and variants of this sequence, have been found to have binding affinity for delta PKC and to induce activation of delta PKC. Thus, in one embodiment, the invention encompasses an isolated peptide comprising the amino acid sequence of SEQ ID NO:1, variants of SEQ ID NO:1, and modifications of SEQ ID NO:1. Preferred variants are peptides having at least about or greater than 80%, and more preferably at least about or greater than 90%, and still more preferably at least about or greater than 95% amino acid sequence identity SEQ ID NO:1, and which retain at least one biological, immunological, or other functional characteristic or activity of SEQ ID NO:1. Studies conducted to identify these sequences and their interaction with delta PKC will now be described.

### Annexin V-Delta PKC Interaction

Initial studies were designed to evaluate binding of annexin V to delta PKC, to certain regions of delta PKC (V1 and V5 regions), and to epsilon PKC. In a study detailed in Example 1, an overlay assay using immobilized annexin V (SEQ ID NO:2) and recombinant full-length delta PKC (SEQ ID NO:3), purified full-length epsilon PKC (SEQ ID NO:6), the recombinant V1 region of delta PKC (“V1 delta PKC”, SEQ ID NO:4), and the recombinant V5 region of delta PKC (“V5 delta PKC”, SEQ ID NO:5) was done. The V1 delta PKC peptide conjugated to myelin basic protein (MBP) to form a fusion protein, and the V5 delta PKC peptide was conjugated to glutathione-S-transferase to form a fusion protein. The results are shown in FIG. 1 and indicate that the full-length delta PKC as well as its V1 and V5 domains bound to annexin V. In contrast, epsilon PKC did not bind to annexin V, indicating the isozyme selectivity of this interaction.

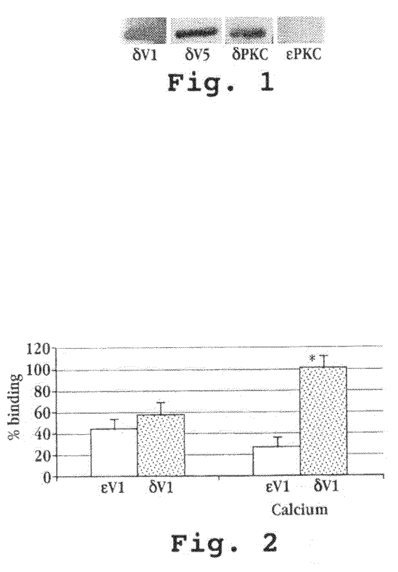

In another study, binding of annexin V protein to the V1 regions of epsilon PKC (SEQ ID NO:7) and of delta PKC (SEQ ID NO:4) was examined using ELISA, as described in Example 2. FIG. 2 shows the percent binding of the indicated domains, epsilon V1 PKC and delta V1 PKC, to annexin V (SEQ ID NO:2), in the presence and absence of calcium, annexin V activator. Binding of annexin V protein (SEQ ID NO:2) to delta V1 PKC in the absence of calcium was about 55%. In the presence of calcium binding was increased to 100%. The extent of annexin V protein binding to epsilon V1 PKC in the presence of calcium was not statistically different from the extent of binding in the absence of calcium. Thus, annexin V protein binds to full-length PCK and to the V1 and V5 domains of delta PKC in vitro in an isozyme specific manner.

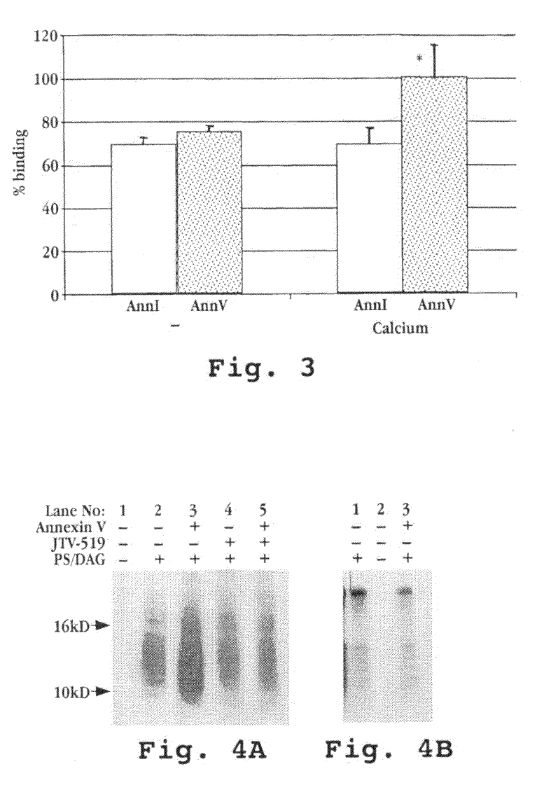

Annexin V binds with higher affinity to delta PKC than does annexin I (SEQ ID NO:9), as was shown in another study presented in FIG. 3. The percent binding of annexin 1 and annexin V to delta PKC (SEQ ID NO:3) in the presence and absence of calcium was investigated using ELISA (Example 2). Annexin V protein (SEQ ID NO:2) in the absence of calcium had a binding of about 75% to delta PKC (SEQ ID NO:3). In the presence of calcium the binding increased to 100%. In contrast, annexin I binding to delta PKC was unaffected by the presence of calcium and remained at about 70% both in the absence and presence of calcium.

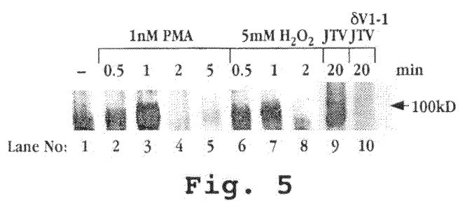

In another study, the ability of annexin V to increase the catalytic activity of delta PKC in vitro was evaluated. Full-length, recombinant delta PKC (SEQ ID NO:3) and epsilon PKC (SEQ ID NO:6) were separately incubated in the presence of kinase buffer, ATP, or histone, as described in Example 3. Activators of PKC, phosphatidylserine (PS) and diacylglycerol (DAG), were added along with annexin V, JTV-519, a 1,4 benzothiazepine derivative that activates delta PKC (Inagaki, K. et al., Circulation, 101:797 (2000)), or a mixture of annexin V and JTV-519. The extent of histone phosphorylation was assessed by SDS-PAGE and the results are shown in FIGS. 4A-4B.

FIG. 4A is an SDS-PAGE gel of delta PKC alone (Lane 1) or in the presence of phosphatidylserine (PS) and diacylglycerol (DAG; Lane 2). PS/DAG stimulates delta PKC, and results in phosphorylation of histone (Lane 2). The addition of annexin V to delta PKC in the presence of PS/DAG (Lane 3) increases the catalytic activity of delta PKC. The addition of JTV-519 to delta PKC in the presence of PS/DAG (Lane 4) results in phosphorylation of histone to about the same extent as observed for delta PKC in the presence of PS/DAG alone (Lane 2). Addition of JTV-519 to delta PKC in the presence of annexin V and PS/DAG (Lane 5) blocked the increased catalytic activity observed with annexin V (Lane 3).

FIG. 4B shows the results for epsilon PKC alone (Lane 2) and in the presence of phosphatidylserine (PS) and diacylglycerol (DAG; Lane 1), and in the presence of PS/DAG and annexin V (Lane 3). As seen, annexin V does not increase the activity of epsilon PKC.

The results in FIGS. 4A-4B show that annexin V selectively increases catalytic activity of delta PKC, and that this increase is blocked by JTV-519.

Example 4 describes a study where cardiac myocytes were stimulated with a selected agent and then immuno-precipitated with anti-delta PKC after cross-linking. The cell lysates were then probed with anti-annexin V and visualized with ECL Western blotting. The agents selected to stimulate the cardiomyocytes were phorbol 12-myristate 13-acetate (PMA), hydrogen peroxide (H₂O₂), JTV-519, and a combination of a peptide inhibitor of delta PKC (delta V1-1 peptide, SEQ ID NO:28) and JTV-519. The stimulation agent was applied to the cells for times of between 30 seconds to 20 minutes, prior to lysing. The results are shown in FIG. 5. Annexin V interacts with delta PKC early after stimulation with PMA (see Lanes 2 and 3 corresponding to 30 seconds and 1 minute of PMA stimulation). The same is observed for cells stimulated with H₂O₂, where 30 seconds and 1 minute of exposure (Lanes 6 and 7, respectively) stimulated delta PKC to a greater extent than exposure for 2 minutes (Lane 8). Interaction of annexin V and delta PKC was greatly enhanced by the presence of JTV-519 (Lane 9). The cross-linked band at 100 kD seen in Lane 8 corresponds to the sum of delta PKC (73 kD) and annexin V (35 kD). However, the interaction of annexin V and delta PKC was blocked when the cells were pre-treated with delta V1-1 peptide for 20 minutes, and then treated with JTV-519 for 20 minutes (Lane 10).

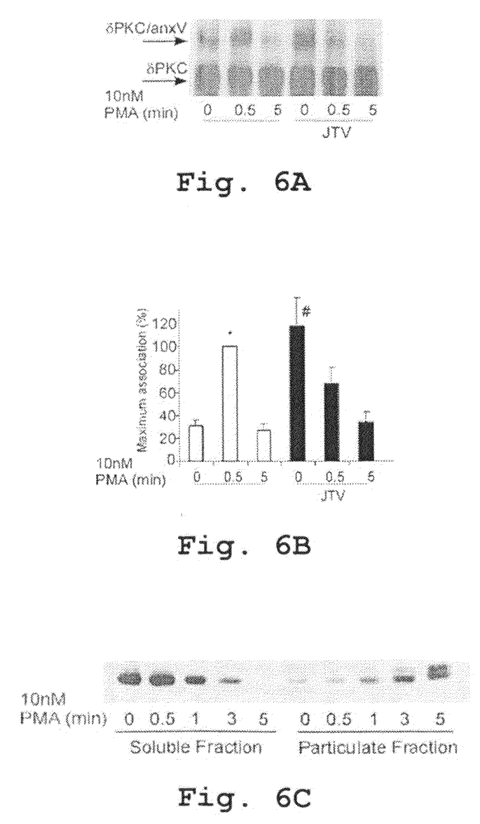

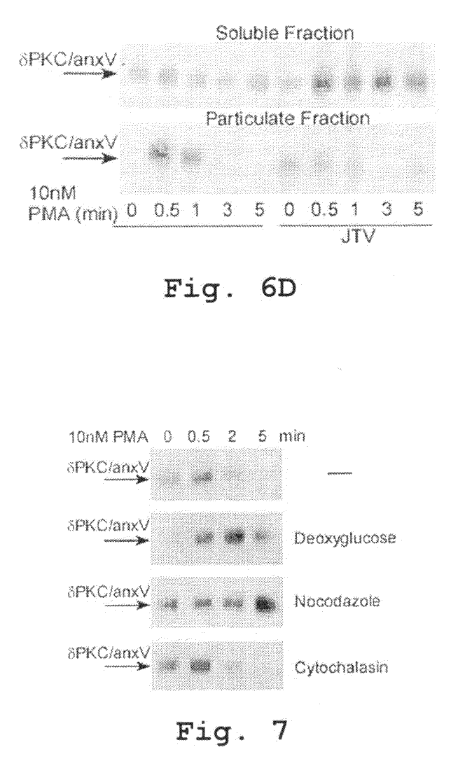

A similar study was conducted to further evaluate the in vitro, intracellular interactions of delta PKC and annexin V. Cellular interactions of delta PKC/annexin V interaction in CHO cells were stabilized with a cross-linking agent, after stimulation with 10 nM PMA. The complexes formed with delta PKC were then immuno-precipitated and tested for the presence of annexin V. The SDS-PAGE gel is shown in FIG. 6A and a corresponding graph quantifying percent association of annexin V and delta PKC as a function of time is shown in FIG. 6B. The figures show that stimulation with PMA, a non-selective PKC activator, induced annexin V and delta PKC interaction within the first 30 seconds of treatment. The interaction is relatively short-lived, and the proteins disassociated after 1 minute of stimulation.

The cross-linked fractionated cell lysates were probed with anti-delta PKC, the SDS-PAGE gel shown in FIG. 6C. Translocation of delta PKC was not observed to occur until after the two proteins disassociate, after 1 minute of PMA stimulation, indicating that association of annexin V and δPKC is upstream of δPKC translocation. Pulldown with anti-epsilon PKC antibodies did not indicate the presence of the εPKC-annexin V complex (data not shown), again demonstrating isozyme specificity.

The effect of JTV-519, an activator of delta PKC, on the association of annexin V and delta PKC was also evaluated in this study. Cells were treated with JTV-519, and the association of annexin V and delta PKC was examined using the co-immunoprecipitation procedure described above. As shown in FIGS. 6A-B, JTV-519 causes full association of annexin V and δPKC at basal conditions, similar to the maximum interaction seen at 30 seconds of PMA stimulation. PMA caused the disassociation of the JTV-induced complex at 30 seconds, the time of maximal PMA-induced association. A closer evaluation of the cellular localization of the delta PKC-annexin V complex revealed that stimulation with PMA caused maximum complex accumulation in the particulate fraction of cells, as seen in FIG. 6D. Whereas the cytosolic fraction of the cells contained a steady low level of the complex, the majority of the complex was found in the particulate fraction of the cells, and disappearance from the particulate fraction followed the same time-course as observed in whole cell lysates (compare FIG. 6D bottom panel with FIG. 6B). After JTV-519 treatment, most of the δPKC-annexin V complex remained in the soluble fraction, with very little in observed in the cell particulate fraction. Thus, formation of the delta PKC-annexin V complex precedes δPKC translocation, and although JTV-519 causes a pre-association of the delta PKC-annexin V complex, it does not allow its proper localization.

To determine the requirements for complex disassociation and subsequent translocation, PMA-induced formation of the delta PKC-annexin V complex was conducted under energetically limiting conditions caused by ATP depletion using 2-deoxyglucose (Kondo, T. et al., J. Lab. Clin. Med., 94:617-23 (1979)), and with cytoskeletal perturbations by pretreatment with actin depolarizing agent cytochalasin D or microtubule-destabilizing agent nocodazole. Experimental details are given in Example 5 and results are shown in FIG. 7.

FIG. 7 shows a series of SDS-PAGE gels illustrating dissociation of delta-PKC and annexin V complex in the presence of deoxyglucose, nocodazole, and cytochalasin. In native conditions (37° C.) the disassociation of the complex was completed by 1 minute of treatment with 10 nM PMA. However, there were substantial levels of the complex even after 5 minutes of PMA stimulation when the cells were treated with 2-deoxyglucose or nocodazole. In contrast, cytochalasin D did not affect the profile of disassociation of the δPKC-annexin V complex. These data suggest that the initial step of delta PKC translocation through its association with annexin V is energy independent. However, the disassociation of the complex requires intact microtubule filaments and appears to be an ATP-dependent process.

Collectively, the data described above shows that annexin V interacts with delta PKC in an isozyme dependent manner. This interaction is calcium dependent and is promoted by JTV-591, a small molecule that interacts with annexin V. The interaction of delta PKC and annexin V is inhibited by delta V1-1, a delta-PKC specific inhibitor. Annexin V increases delta PKC catalytic activity, and JTV-519 prevents the annexin-dependent increase in its activity. The data also shows that annexin V and delta PKC interact at early timepoints of stimulation.

### Annexin V Peptides

Analysis of the annexin V protein sequence (SEQ ID NO:2), the delta PKC sequence (SEQ ID NO:3), and the pseudo-delta RACK sequence (SEQ ID NO:10, U.S. Pat. No. 6,855,693) revealed a sequence LQANRDPD (SEQ ID NO:1, residues 157-164 of annexin V) that is present in annexin V (SEQ ID NO:2), but not present in other annexin proteins. Binding of the annexin V peptide (SEQ ID NO: 1) and of variants of the peptide to delta PKC was examined using an ELISA protocol, detailed in Example 6. Delta PKC (SEQ ID NO:3) was immobilized in the wells of a microtiter plate and then incubated with the annexin V peptide conjugated to alkaline phosphatase or with variants of the annexin V peptide, also conjugated to alkaline phosphatase.

The variants of the annexin V peptide (SEQ ID NO: 1) synthesized for use in the study included LQANRDP (SEQ ID NO: 11) MQMRDP (SEQ ID NO: 12), MRAAENP (SEQ ID NO:13), MRMQDP (SEQ ID NO:14), MEMEDP (SEQ ID NO:15), MRAAADP (SEQ ID NO:16), MAAAEDP (SEQ ID NO:17), MRAAEDP (SEQ ID NO:18). Relative binding is shown in Table 1 by “+” and “−” indicators.

TABLE 1

| | | |
|---|---|---|
| Peptide Sequence | SEQ ID NO. | Binding |

| | | |
|---|---|---|
| MQAARDP | 12 | +++ |
| MRAAENP | 13 | +++ |
| MRAAQDP | 14 | ++ |
| MEAAEDP | 15 | + |
| MRAAADP | 16 | −−− |
| MAAAEDP | 17 | − |
| MRAAEDP | 18 | ++ |

This data shows that the peptide variants of SEQ ID NO:1, specifically, peptides MQAARDP (SEQ ID NO:12), MRMENP (SEQ ID NO:13), MRMQDP (SEQ ID NO:14), and MEMEDP (SEQ ID NO:15) bind to delta PKC. In particular, these variants bind to delta PKC with higher affinity than similar sequences that reverse or eliminate (e.g., SEQ ID NOs:16, 17) the charge of the annexin V peptide.

Other variants contemplated include the sequences identified herein as SEQ ID NO:19 (MQMRDPD), SEQ ID NO:20 (MRAAENPD), SEQ ID NO:21 (MRAAQDPD), SEQ ID NO:22 (MEAAEDPD), SEQ ID NO:23 (MRAAADPD), SEQ ID NO:24 (MAAAEDPD), and SEQ ID NO:25 (MRAAEDPD).

The ability of the annexin V peptide to block the intracellular interaction of delta PKC and annexin V was illustrated in the following study. The annexin V peptide (SEQ ID NO: 1) was conjugated to a TAT₄₇₋₅₇ carrier peptide (SEQ ID NO:26) through an N-terminal Cys-Cys bond, as previously described (Chen, L. et al., Proc. Natl. Acad. Sci. USA, 98:11114-9 ((2001)). This peptide, identified herein as SEQ ID NO:29 and referred to as TAT-AnxV peptide, was used to pretreat (1 μM for 15 minutes) CHO cells prior to stimulation with PMA (10 nM for 5 minutes). For comparison, the annexin V peptide sequence was modified at the Arg (R) residue with a Glu (E) residue, to form a peptide having the sequence LQANEDPD, identified herein as SEQ ID NO:30. Substitution of E for R confers a charge difference between annexin V and delta PKC. This peptide was also attached to a TAT carrier peptide via N-terminal modification with a Cys-Cys bond. The conjugate is referred to herein as TAT-AnxV_{(R→E)} (SEQ ID NO:31) and is denoted in some figures as “EpAnxV.” Cells were similarly pretreated with TAT-AnxV_{(R→E)}, prior to stimulation with PMA. As controls, some cells were left untreated and other cells were exposed only to PMA.

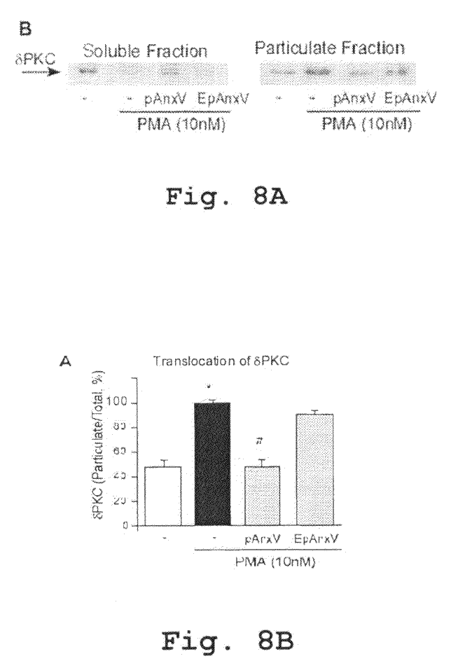

An SDS-PAGE gel showing the soluble and particulate cellular fractions for the four cell populations is shown in FIG. 8A, and a corresponding bar graph showing the fraction of delta PKC protein present in the particulate (membrane) fraction relative to the total protein is shown in FIG. 8B. Untreated cells are indicated along the x-axis with a dash (open bars), and cells treated only with PMA by a dash and the line indicating PMA exposure. The TAT-AnxV peptide blocked the translocation of delta PKC to the cell membrane upon PMA stimulation. The modified peptide, TAT-AnxV_{(R→E)}, lost its inhibitory effect on delta PKC translocation, indicating that delta PKC interaction with annexin V is critical and specific for delta PKC translocation.

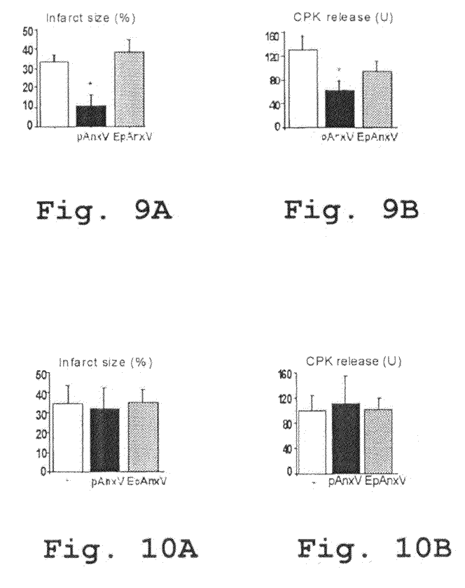

The physiologic impact of disruption of the delta PKC-annexin V complex was evaluated in an ex vivo ischemia/reperfusion study using the annexin V peptide, TAT-AnxV, and its mutant, TAT-AnxV_{(R→E)}. As described in Example 7, isolated rat hearts were treated with TAT-AnxV or TAT-AnxV_{(R→E)} for 10 minutes prior to induction of an ischemic episode. The ischemic episode lasted for 30 minutes, at which time flow to the heart was returned for 60 minutes (reperfusion). After the reperfusion period, cardiac effluent was collected and analyzed for creatine phosphokinase (CPK). The hearts were stained and visually inspected for live and dead tissue. Infarct size was measured by taking the average of the dead (white) tissue on both sides of the tissue slice. The results are shown in FIGS. 9A-9B.

FIG. 9A is a bar graph showing the infarct size, in percent, in the isolated rat hearts subjected to ischemia and reperfusion, and left untreated or treated prior to ischemia with TAT-AnxV peptide or TAT-AnxV_{(R→E)}. Hearts treated with the annexin V peptide were protected from tissue damage, as evidenced by the statistically significant decrease in infarct size in the TAT-AnxV peptide treated hearts. Hearts treated with TAT-AnxV_{(R→E)} did not inhibit the delta PKC-induced damage to the heart tissue. The protective effects of the TAT-AnxV peptide when provided prior to an ischemic episode is also observed by the reduced level of creatine phosphokinase (CPK) in hearts treated with the peptide, as seen in FIG. 9B.

A similar study was conducted, except rather than pretreating the isolated hearts with the annexin V peptide, the peptide was administered after the ischemic event and during the initial 10 minutes of the reperfusion period. Hearts were similarly treated with the mutant peptide, TAT-AnxV_{(R→E)}. The infarct size and CPK release from the hearts are shown in FIGS. 10A-10B, respectively. As seen in FIG. 10A, when the annexin V peptide was applied only at the onset of reperfusion, no cardioprotective effect was observed, as evidenced by the measured infarct size of the treated and untreated hearts being essentially the same. The CPK levels, shown in FIG. 10B, also indicate that the annexin V peptide does not confer a cardioprotective effect when given at the onset of reperfusion. This observation is consistent with the data above indicating that delta PKC/annexin V interaction occurs early upon activation of delta PKC, and that to inhibit delta PKC translocation, the annexin V inhibitor peptide is needed during signal initiation. These data indicate that inhibition of delta PKC-annexin V interaction is an essential step in delta PKC function, and confirm that the formation of delta PKC-annexin V complex occurs early in the activation pathway of delta PKC.

In summary, the data illustrates a protein-protein interaction that precedes PKC translocation. This interaction is selective for delta PKC and annexin V. Thus, in one embodiment, a substantially pure, isolated or recombinant polypeptide is provided, which is selected from the group consisting of: (i) the amino acid sequence comprising or consisting of the sequences identified herein as SEQ ID NO: 1 or SEQ ID NO: 11; and (ii) a variant having one or more amino acid substitutions, modifications, deletions, or insertions relative to the amino acid sequence identified as SEQ ID NO:1, the variant being at least about 50% identical to SEQ ID NO:1 and retaining at least a proportion of the activity of SEQ ID NO:1. The peptide acts to inhibit translocation of delta PKC, by inhibiting the association of delta PKC with annexin V, thereby conferring a protective effect to cells at risk of exposure to ischemia.

A peptide within the scope of (i) above may consist of the particular amino acid sequence identified as SEQ ID NO:1 or SEQ ID NO:11, or may have an additional N-terminal and/or an additional C-terminal amino acid sequence relative to the sequences identified as SEQ ID NO:1 and SEQ ID NO:11.

Peptides within the scope of (ii) above are peptides which are variants of the peptide identified by SEQ ID NO:1, provided that such variant exhibits at least a proportion of the biological activity of the peptide identified by SEQ ID NO: 1 and is at least about 50% identical to the peptide identified by SEQ ID NO: 1. It will be appreciated that alterations in the amino acid sequence of a peptide can occur which do not affect the function of a peptide, as illustrated with the peptides identified as SEQ ID NO:12, 13, and 14 (Table 1). These include amino acid deletions, insertions, modifications, and substitutions and can result from alternative splicing and/or the presence of multiple translation start sites and stop sites. Polymorphisms may arise as a result of the infidelity of the translation process. Thus changes in amino acid sequence may be tolerated which do not affect the protein's function.

The skilled person will appreciate that various changes can often be made to the amino acid sequence of a polypeptide which has a particular activity to produce variants (sometimes known as derivatives or “muteins”) having at least a proportion of the desired biological activity, and preferably having a substantial proportion of said activity. The skilled person is aware that various amino acids have similar properties. One or more such amino acids of a substance can often be substituted by one or more other such amino acids without eliminating a desired activity of that substance. For example, the amino acids glycine, alanine, valine, leucine and isoleucine can often be substituted for one another (amino acids having aliphatic side chains). Of these possible substitutions, it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur-containing side chains). Substitutions of this nature are often referred to as “conservative” or “semi-conservative” amino acid substitutions.

Amino acid deletions or insertions may also be made relative to the amino acid sequence identified as SEQ ID NO:1. Thus, for example, amino acids which do not have a substantial effect on the activity of the peptide, or at least which do not eliminate such activity, may be deleted. Such deletions can be advantageous since the overall length and the molecular weight of a peptide can be reduced whilst still retaining activity. This can enable the amount of peptide required for a particular purpose to be reduced—for example, dosage levels can be reduced.

Whatever amino acid changes are made (whether by means of substitution, insertion, or deletion), preferred peptides of the present invention have at least 50% sequence identity with the peptide identified as SEQ ID NO:1, more preferably the degree of sequence identity is at least 75%. Sequence identities of at least 80%, at least 85%, at least 90%, at least 95% or at least 98% are most preferred.

The annexin V peptide and the described variants of the annexin V peptide may be produced using chemical methods to synthesize the amino acid sequence. For example, peptide synthesis can be performed using various solid-phase techniques (Roberge, J. Y. et al., Science, 269:202 (1995)) and automated synthesis may be achieved, for example, using the ABI 431 A Peptide Synthesizer (Perkin Elmer). The synthesized peptide may be substantially purified by preparative high performance liquid chromatography (e.g., Creighton, T. (1983) Proteins, Structures and Molecular Principles, WH Freeman and Co., New York, N.Y.). The composition of the synthetic peptides may be confirmed by amino acid analysis or sequencing (e.g., the Edman degradation procedure; Creighton, supra). Additionally, the amino acid sequence of the annexin V peptide, or any part thereof, may be altered during direct synthesis and/or combined using chemical methods with sequences from other proteins, or any part thereof, to produce a variant polypeptide.

The invention also contemplates a modified annexin V peptide, where the annexin V peptide and any of the variants is extended on the C-terminus or N-terminus by a number of amino acids. A preferred modified annexin for use in the present invention is extended on the N-terminus with an amino acid residue that provides an accessible sulfhydryl group. Such a modified annexin can be conjugated to a any number of carrier peptides to assist in transport of the annexin V peptide or annexin V peptide variant across a cell membrane. For example, the annexin V peptide can include one or more cysteine residues added to the N-terminus or to the C-terminus. The sulfhydryl group or groups of the added cysteine residue or residues can be joined to known carrier peptides, such as TAT (SEQ ID NO:26) or to Drosophila Antennapedia homeodomain (SEQ ID NO:27; Théodore, L. et al. J. Neurosci., 15:7158 (1995); Johnson, J. A. et al., Circ. Res. 79:1086 (1996)) or to polyarginine (Mitchell et al., J. Peptide Res., 56:318-325 (2000); Rothbard et al., Nature Med., 6:1253-1257 (2000)).

It will be appreciated that the invention also encompasses polynucleotides which encode the peptide set forth as SEQ ID NO:1 and which encode variants of SEQ ID NO: 1. Accordingly, any nucleic acid sequence which encodes the amino acid sequence LQANRDPD can be used to produce recombinant molecules which express LQANRDPD. It will be appreciated by those skilled in the art that as a result of the degeneracy of the genetic code, a multitude of nucleotide sequences encoding SEQ ID NO: 1, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code, and all such variations are to be considered as being specifically disclosed.

### III. Compositions and Methods of Treatment

The invention also contemplates a method of regulating the delta PKC-mediated cellular responses by administering an annexin V peptide having a sequence corresponding to SEQ ID NO:1 or a sequence having greater than or at least about 50% identity to SEQ ID NO:1, more preferably 75% identity, still more, preferably 80%, 85%, 90%, or 95% identity to SEQ ID NO:1. In the method, the peptide is administered in a suitable formulation, where the peptide is effective to bind to delta PKC and modulate interaction between annexin V and delta PKC in vivo.

The invention also contemplates a method of protecting tissue against damage due to ischemia or due to reperfusion injury. In this method, a patient at risk of ischemia is treated with an annexin V peptide (SEQ ID NO: 1) or a variant or a modification thereof, to induce preconditioning and protection against ischemia.

A pharmaceutical composition comprising the annexin V peptide or a variant thereof is also contemplated. The pharmaceutical composition comprises the peptide in combination with a carrier, selected in part according to the desired route of administration. Any number of routes of administration are contemplated, including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

Suitable pharmaceutically-acceptable carriers comprise excipients and auxiliaries which facilitate processing of the active compounds into preparations. Further details on techniques for formulation and administration may be found in the latest edition of Remington's Pharmaceutical Sciences (Maack Publishing Co., Easton, Pa.). For example, pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

After pharmaceutical compositions have been prepared, they can be placed in an appropriate container and labeled for treatment of an indicated condition. For administration of the annexin V peptide, such labeling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For example, the therapeutically effective dose can be estimated initially either in cell culture assays or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example the annexin V peptide or variant thereof, which ameliorates the symptoms or the condition. The data obtained from cell culture assays and animal studies is used in formulating a range of dosage for human use. The dosage contained in such compositions is preferably within a range of circulating concentrations that provides therapeutic efficacy with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Exemplary therapeutically effective amounts of peptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. Treatment of a subject with a therapeutically effective amount of the peptide can include a single treatment or, preferably, can include a series of treatments.

### IV. EXAMPLES

The following examples further illustrate the invention described herein and are in no way intended to limit the scope of the invention.

### Example 1

### Overlay Assay Showing that Human Annexin V Binds to Delta PKC

10 μg of annexin V (SEQ ID NO:2) was separated on 12% SDS-PAGE gel and transferred to nitrocellulose, and then cut into strips. The strips were incubated with blocking buffer (50 mM Tris pH 7.5, 200 mM NaCl, 3% bovine serum albumin, 0.1% PEG20). The strips were then incubated in overlay buffer (50 mM Tris pH 7.5, 200 mM NaCl, 1% PEG20, 12 mM β-mercaptoethanol) in the presence where appropriate of 30 μg crude bacterial cell lysate containing fusion proteins of myelin basic protein (MBP)-delta V1 PKC (MBP-δV1), glutathione-S-transferase-delta V5 PKC (GST-δV5), and full length sequences of delta (δPKC, SEQ ID NO:3) or rat purified epsilon (εPKC, SEQ ID NO:6) at 37° C. for 30 min. The strips where then washed with wash buffer (50 mM Tris pH 7.5, 200 mM NaCl, 0.1% PEG20, 12 mM O-mercaptoethanol), and then incubated with fixative (0.2% formaldehyde in PBS) and neutralizing solution (0.2M glycine in PBS) at room temperature 20 min each. After washing with buffer, the strips were probed with antibodies according to the overlay protein, and resulting bands visualized using ECL Western blotting. The results are shown in FIG. 1.

### Example 2

### Analysis of Binding of Delta PKC Domains to Annexin V

A. ELISA Binding Assay

Annexin V (SEQ ID NO:2) was diluted in 100 mM carbonate buffer (pH=9.6) and 100 μl of the diluted solution is placed in each well of a microtiter plate to give 0.1 μg Annexin V per well. The plate was incubated overnight at 4° C. After the overnight incubation, the plate was blocked for 1 hour at room temperature with 200 μL/well of 1% bovine serum albumin (BSA).

The interaction partners, delta PKC (SEQ ID NO:3), delta V1 PKC (SEQ ID NO:4), and delta V5 PKC (SEQ ID NO:5), were diluted in 100 mM HEPES. For the full length delta PKC, approximately 1.5 μg/well (1:20 dilution of cell lysate) was added. For the delta V1 and delta V5 PKC regions, between about 8000-80 ng/well was added, with an intermediate concentration being sufficient. Signal to noise increased dramatically at higher concentrations; therefore, 8 μg/well was preferred (this corresponds to 1:40 dilution of cell lysate). 100 μL/well, 1 hour w/shaking. Binding was accelerated at 37° C., however room temperature was also sufficient for binding.

After 1 hour shaking, the plate was washed three times with 100 mM HEPES (200 μL/well×3), with 5 minutes for each wash, with shaking.

Next, a primary antibody in milk (usually 1:1000 dilution) was added, followed by 1 hour at room temperature with shaking. The plate was then washed three times, (200 μL/well×3) with PBS Tween (0.1% Tween), with 5 minutes per wash with shaking.

Then, the secondary antibody diluted in PBS Tween (usually 1:1000) was added, followed by 1 hour at room temperature with shaking. The plate was then washed three times (200 μL/well×3) with PBS Tween, with 5 minutes per wash with shaking.

A developing reagent was added (100 μL/well): 1 PNPP tablet per 5 mL diluted buffer. 2N NaOH was added and the plate was allowed to develop and then read at 405 nm (single wavelength). The results are shown in FIG. 2.

### Example 3

### Kinase Assay

200 ng of pure delta PKC or epsilon PKC (Panvera LLC, now Invitrogen Corporation, Carlsbad, Calif.) were incubated in 100 μL kinase buffer (20 mM Tris pH 7.4, 50 mM MgCl₂, 0.3 μCi/reaction [γ-³²P]ATP (Amersham Biosciences), 9 μM/reaction ATP (Sigma), 30 μg/reaction myelin basic protein or histone A1 (Sigma); 100 μM lipid vesicle mixture (90% phosphatidylserine (PS): 10% diacylglycerol (DAG)), 1 mM CaCl₂, 1 μM JTV519, and/or 20 ng of Annexin V/reaction were added, where applicable. The kinase reaction was stopped by the addition of SDS sample buffer, and extent of histone phosphorylation assessed after separation on 15% SDS-PAGE and transfer onto nitrocellulose membrane by autoradiography. The results are shown in FIGS. 4A-4B.

### Example 4

### Cross-Linking Immuno-Precipitation

Cardiomyocytes were treated with phorbol 12-myristate 13-acetate (PMA; 10 nM), hydrogen peroxide (H₂O₂; 5 mM), JTV-519, or delta V1-1 peptide (1 μM) and JTV-519 (1 μM), for times of between 30 seconds to 5 minutes. After the lapsed time, the cells were washed with cold PBS, and homogenized on ice with trituration in homogenization buffer (20 mM Tris pH 7.4, 2 mM EDTA, 10 mM EGTA, 0.25M sucrose, 12 mM O-mercaptoethanol, and protease inhibitor cocktail (Sigma), 0.1% formaldehyde. After 30 min at 4° C., the lysates were quenched with 0.14M glycine for 20 min at 4° C., and samples spun at 14K rpm at 4° C. The supernatant was incubated with 1 μg anti-delta PKC antibody (Santa Cruz Biotechnology, Inc., Santa Cruz, Calif.) for 1 hour, then with proteinG beads (Invitrogen Corporation) for 3 hours at 4° C. The beads where then washed with wash buffer (20 mM Tris pH 7.5, 2 mM EDTA, 100 mM NaCl, 12 mM β-mercaptoethanol, 0.1% Triton), and separated on 7.5% SDS-PAGE, transferred to nitrocellulose, and probed with anti-annexin V (Santa Cruz Biotechnology, Inc., Santa Cruz, Calif.), followed by visualization by ECL Western blotting. The results are shown in FIG. 5.

### Example 5

### Dissociation of Delta-PKC-Annexin V Complex

CHO cells were pretreated with one of the following: (1) 2-deoxyglucose (20 mM) for 4 hours in a glucose-free buffer; (2) nocodazole (10 μM) for 2 hours; or (3) cytochalasin D (2 μM) for 1 hour. The cells were then treated with phorbol myristate acetate (PMA; 10 nM) for times of between 30 seconds to 5 minutes. After the lapsed time, the cells were washed with cold PBS, and homogenized on ice with trituration in homogenization buffer (20 mM Tris pH 7.4, 2 mM EDTA, 10 mM EGTA, 0.25M sucrose, 12 mM β-mercaptoethanol, and protease inhibitor cocktail (Sigma), 0.1% formaldehyde. After 30 min at 4° C., the lysates were quenched with 0.14M glycine for 20 min at 4° C., and samples spun at 14K rpm at 4° C. The supernatant was incubated with 1 μg anti-delta PKC antibody (Santa Cruz Biotechnology, Inc., Santa Cruz, Calif.) for 1 hour, then with proteinG beads (Invitrogen Corporation) for 3 hours at 4° C. The beads where then washed with wash buffer (20 mM Tris pH 7.5, 2 mM EDTA, 100 mM NaCl, 12 mM β-mercaptoethanol, 0.1% Triton), and separated on 7.5% SDS-PAGE, transferred to nitrocellulose, and probed with anti-annexin V (Santa Cruz Biotechnology, Inc., Santa Cruz, Calif.), followed by visualization by ECL Western blotting. The results are shown in FIG. 7.

### Example 6

### Elisa Protocol for Annexin V Peptides Fused to Alkaline Phosphotase

Recombinant delta PKC protein (SEQ ID NO:3) was bound to a Falcon plate for 1 hour at room temperature in carbonate buffer pH 9.6. A 1:10-1:100 dilution of PKC enzyme from glycerol stock or 100 μg was prepared. β-mercaptoethanol at 1 μL/mL was added into the PKC. Each well contained approximately 100 μL of PKC; some wells without PKC were present to correct for non-specific binding.

Next, all wells were blocked with 200 μL of super block or 0.5% milk in carbonate buffer. Blocking was done twice, for 5 minutes each time.

Without washing the wells, 100 μL of a peptide fused to alkaline phosphatase protein (i.e., peptide-alkaline phosphatase fusion) was added to the wells, and incubated for 30 minutes at 37° C. The wells were then washed three times, for 2 minutes each time, with TBS/Tween (0.1%). The wells were allowed to develop overnight before reading absorbance at 405 nm. The results are shown in Table 1.

### Example 7

### Ex Vivo Treatment of Hearts with the Annexin V Peptide

Rats were anesthetized and their hearts were rapidly removed and cannulated via the aorta for retrograde perfusion (Colbert, M. et al., J. Clin. Invest., 100:1958 (1997)). The hearts were left untreated, or were pretreated with the annexin V peptide, conjugated to TAT, (TAT-AnxV, SEQ ID NO:29) or with its mutant, TAT-AnxV_{(R→E)} (SEQ ID NO:31), for 10 minutes prior to a 30 minutes of ischemia. Ischemia was induced by interrupting flow for 30 minutes and then reestablishing flow for 60 minutes (reperfusion). Cardiac effluent was collected for 15 minutes after reperfusion and assayed for creatine phosphokinase (CPK) using a Sigma kit. Hearts were stained with 2,3,5-triphenyltetrazolium chloride (UTC) to visualize live tissue (red) and dead tissue (white), and infarct size was measured by taking the average of the white area on both sides of the tissue slice (n=3, p<0.05). Results are shown in FIGS. 9A-9B and FIGS. 10A-10B.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A purified peptide having at least 50% amino acid identity over the complete sequence of LQANRDPD (SEQ ID NO:1) and which possesses delta-protein kinase C (PKC) binding activity.

2. A composition comprising a peptide having at least 50% amino acid identity over the complete sequence of LQANRDPD (SEQ ID NO:1) and which possesses delta-PKC binding activity and a pharmaceutically acceptable carrier.

3. A purified peptide comprising an amino acid sequence of LQANRDPD (SEQ ID NO:1).

4. A peptide or composition according to claim 1 further including one or more C-terminal or N-terminal cysteine resides.

5. The peptide of claim 4, modified to a cell transport carrier moiety.

6. Use of the peptide according to claim 1 for regulating a cellular response mediated by delta PKC or protecting tissue from ischemia or hypoxia.

7. A method for regulating a cellular response mediated by delta PKC or for protecting tissue from damage due to ischemia or reperfusion, comprising
administering a composition comprised of a peptide having at least 50% amino acid identity over the complete sequence of LQANRDPD (SEQ ID NO:1) and which possesses delta-PKC binding activity, and a pharmaceutically-acceptable carrier.

8. The method according to claim 7 wherein the peptide further includes one or more C-terminal or N-terminal cysteine resides.

9. The method according to claim 8, wherein the peptide is modified to a cell transport carrier moiety.

10. A peptide or composition according to claim 2 further including one or more C-terminal or N-terminal cysteine resides.

11. A peptide or composition according to claim 3 further including one or more C-terminal or N-terminal cysteine resides.

12. A peptide or composition according to claim 10 further including one or more C-terminal or N-terminal cysteine resides.

13. A peptide or composition according to claim 11 further including one or more C-terminal or N-terminal cysteine resides.

14. Use of the peptide according to claim 2 for regulating a cellular response mediated by delta PKC or protecting tissue from ischemia or hypoxia.

15. Use of the peptide according to claim 3 for regulating a cellular response mediated by delta PKC or protecting tissue from ischemia or hypoxia.

16. Use of the peptide according to claim 4 for regulating a cellular response mediated by delta PKC or protecting tissue from ischemia or hypoxia.

17. Use of the peptide according to claim 5 for regulating a cellular response mediated by delta PKC or protecting tissue from ischemia or hypoxia.


SEQUENCE LISTING (31 sequences)

SEQ ID NO: 1 - protein, 8 aa, Homo sapiens
LQANRDPD

SEQ ID NO: 2 - protein, 318 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSKMIKGDTSGDYKKALLLLCGEDD

SEQ ID NO: 3 - protein, 672 aa, Rattus norvegicus
MAPFLRISFNSYELGSLQVEDEASQPFCAVKMKEALSTERGKTLVQKKPTMYPEWKTTFDAHIYEGRVIQIVLMRAAEDPVSEVTVGVSVLAERCKKNNGKAEFWLDLQPQAKVLMCVQYFLEDGDCKQSMRSEEEAKFPTMNRRGAIKQAKIHYIKNHEFIATFFGQPTFCSVCKEFVWQKQGYKCRQCNAAIHKKCIDKIIGRCTGTATNSRDTIFQKERFNIDMPHRFKVYNYMSPTFCDHCGSLLWGLVKQGLKCEDCGMNVHHKCREKVANLCGINQKLLAEALNQVTQRSSRKLDTTESVGIYQGFEKKPEVSGSDILDNNGTYGKIWEGSTRCTLENFTFQKVLGKGSFGKVLLAELKGKDKYFAIKCLKKDVVLIDDDVECTMVEKRVLALAWESPFLTHLICTFQTKDHLFFVMEFLNGGDLMFHIQDKGRFELYRATFYAAEIICGLQFLHSKGIIYRDLKLDNVMLDRDGHIKIADFGMCKENIFGEGRASTFCGTPDYIAPEILQGLKYSFSVDWWSFGVLLYEMLIGQSPFHGDDEDELFESIRVDTPHYPRWITKESKDIMEKLFERDPDKRLGVTGNIRIHPFFKTINWSLLEKRKVEPPFKPKVKSPSDYSNFDPEFLNEKPQLSFSDKNLIDSMDQEAFHGFSFVNPKFEQFLDI

SEQ ID NO: 4 - protein, 160 aa, Rattus norvegicus
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPTMNRRGAIKQAKIHYIKNHE

SEQ ID NO: 5 - protein, 55 aa, Rattus norvegicus
PKVKSPSDYSNFDPEFLNEKPQLSFSDKNLIDSMDQEAFHGFSFVNPKFEQFLDI

SEQ ID NO: 6 - protein, 735 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDRIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 7 - protein, 144 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEE

SEQ ID NO: 8 - protein, 48 aa, Homo sapiens
KTKRDVNNFDQDFTREEPVLTLVDEAIVKQINQEEFKGFSYFGEDLMP

SEQ ID NO: 9 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 10 - protein, 8 aa, Rattus norvegicus
MRAAEDPM

SEQ ID NO: 11 - protein, 7 aa, Artificial Sequence
  variant of SEQ ID NO1
LQANRDP

SEQ ID NO: 12 - protein, 7 aa, Artificial Sequence
  variant of SEQ ID NO1
MQAARDP

SEQ ID NO: 13 - protein, 7 aa, Artificial Sequence
  variant of SEQ ID NO1
MRAAENP

SEQ ID NO: 14 - protein, 7 aa, Artificial Sequence
  variant of SEQ ID NO1
MRAAQDP

SEQ ID NO: 15 - protein, 7 aa, Artificial Sequence
  variant of SEQ ID NO1
MEAAEDP

SEQ ID NO: 16 - protein, 7 aa, Artificial Sequence
  variant of SEQ ID NO1
MRAAADP

SEQ ID NO: 17 - protein, 7 aa, Artificial Sequence
  variant of SEQ ID NO1
MAAAEDP

SEQ ID NO: 18 - protein, 7 aa, Artificial Sequence
  variant of SEQ ID NO1
MRAAEDP

SEQ ID NO: 19 - protein, 8 aa, Artificial Sequence
  variant of SEQ ID NO1
MQAARDPD

SEQ ID NO: 20 - protein, 8 aa, Artificial Sequence
  variant of SEQ ID NO1
MRAAENPD

SEQ ID NO: 21 - protein, 8 aa, Artificial Sequence
  variant of SEQ ID NO1
MRAAQDPD

SEQ ID NO: 22 - protein, 8 aa, Artificial Sequence
  variant of SEQ ID NO1
MEAAEDPD

SEQ ID NO: 23 - protein, 8 aa, Artificial Sequence
  variant of SEQ ID NO1
MRAAADPD

SEQ ID NO: 24 - protein, 8 aa, Artificial Sequence
  variant of SEQ ID NO1
MAAAEDPD

SEQ ID NO: 25 - protein, 8 aa, Artificial Sequence
  variant of SEQ ID NO1
MRAAEDPD

SEQ ID NO: 26 - protein, 17 aa, Artificial Sequence
  Drosophila Antennapedia homeodomain-derived carrier peptide
CRQIKIWFQNRRMKWKK

SEQ ID NO: 27 - protein, 10 aa, Artificial Sequence
  Tat-derived carrier peptide
YGKKRRQRRR

SEQ ID NO: 28 - protein, 9 aa, Rattus norvegicus
SFNSYELGS

SEQ ID NO: 29 - protein, 21 aa, Artificial Sequence
  Synthetic conjugate TAT-AnxV
YGRKKRRQRRRCCLQANRDPD

SEQ ID NO: 30 - protein, 8 aa, Artificial Sequence
  Modification of SEQ ID NO1
LQANEDPD

SEQ ID NO: 31 - protein, 21 aa, Artificial Sequence
  Synthetic conjugate EpAnxV
YGRKKRRQRRRCCLQANEDPD